(19) United States
(12) Patent Application Publication
(10) Pub. No.: US 2025/0143944 A1
(43) Pub. Date: May 8, 2025

(54) Multi-Functional Hyperbaric Oxygen Device With Integrated Remote Mind-Body Synergy System For Enhanced Health Management

(71) Applicant: Xu Fen Tu, Glendora, CA (US)

(72) Inventor: Xu Fen Tu, Glendora, CA (US)

(21) Appl. No.: 19/011,674

(22) Filed: Jan. 7, 2025

Related U.S. Application Data
(60) Provisional application No. 63/694,222, filed on Sep. 13, 2024.

(51) Int. Cl. A61G 10/02; A61M 21/00; A61M 21/02; G16H 15/00; G16H 20/70; G16H 40/63; G16H 80/00

(52) U.S. Cl. CPC A61G 10/026; A61M 21/02; G16H 15/00; G16H 20/70; G16H 40/63; G16H 80/00; A61G 2203/46; A61M 2021/0027; A61M 2021/0044; A61M 2202/0007; A61M 2202/0208; A61M 2205/051; A61M 2205/18; A61M 2205/3327; A61M 2205/3368; A61M 2205/3553; A61M 2205/502; A61M 2205/581; A61M 2205/583; A61M 2230/005; A61M 2230/06; A61M 2230/20; A61M 2230/42; A61M 2230/50

(57) ABSTRACT

The present invention relates to a multi-functional hyperbaric oxygen device that integrates hyperbaric oxygen therapy unit, light therapy unit, temperature regulation unit, and a remote mind-body synergy system for chronic disease recovery, physiological and psychological health improvement, and comprehensive health management. The multi-functional hyperbaric oxygen device allows users to receive personalized, data-driven therapies, adjusting oxygen levels, light wavelengths, and temperature settings based on real-time physiological and psychological monitoring. The multi-functional hyperbaric oxygen device enables users to conduct therapy sessions from the comfort of their home, thereby minimizing the need for frequent visits to specialized medical centers, and improving accessibility for those with mobility challenges. The multi-functional hyperbaric oxygen device ensures patient safety by incorporating automated oxygen regulation, CO₂ management, and safety sensors to prevent risks associated with excessive oxygen exposure and ensure optimal therapeutic conditions.

### FIELD OF THE INVENTION

The present invention relates to medical technology and health management, specifically to a multi-functional hyperbaric oxygen device that integrates hyperbaric oxygen therapy, light therapy, sleep assistance, and temperature regulation with a remote mind-body synergy system for chronic disease recovery, physiological and psychological health improvement, and comprehensive health management.

### BACKGROUND

Hyperbaric oxygen therapy (HBOT) has long been recognized for its therapeutic benefits, particularly in the treatment of chronic wounds, sports injuries, and certain types of infections. HBOT involves the administration of oxygen in a pressurized chamber, which enhances oxygen supply to tissues and accelerates the body's natural healing process. However, while hyperbaric oxygen therapy has proven effective in many clinical applications, its utilization has traditionally been limited to specific medical conditions and clinical environments, often requiring patients to visit specialized treatment centers. Furthermore, the benefits of HBOT are typically focused on physical recovery, with little integration of psychological or mental health support.

Conventional hyperbaric oxygen chambers operate by maintaining high levels of oxygen pressure in a confined space. While these chambers have been effective in treating physical ailments such as wounds and infections, they often lack advanced features that address the holistic needs of patients, such as psychological wellness, chronic stress management, and emotional recovery. As a result, patients undergoing HBOT may experience physical recovery but fail to address underlying psychological factors that impact their overall well-being. Additionally, conventional chambers are usually expensive, large, and require frequent visits to specialized facilities, thereby limiting accessibility for patients in remote areas or those with mobility restrictions.

Another significant limitation of current HBOT technology is its inability to integrate multiple therapeutic modalities, such as light therapy and sleep assistance, into the treatment process. Light therapy, particularly the use of specific wavelengths such as red and near-infrared light, has been shown to promote healing, reduce inflammation, and improve sleep quality. However, existing hyperbaric oxygen chambers do not incorporate such therapies into their design, which leads to missed opportunities to optimize recovery through multi-modal approaches. Furthermore, the lack of sleep-assistive features within traditional chambers leaves many patients vulnerable to sleep disturbances that could hinder the healing process.

While there has been significant progress in the development of remote medical technologies, such as telemedicine and virtual consultations with healthcare professionals, existing systems for remote psychological and physical therapy often suffer from limitations in their integration of real-time physiological monitoring and multi-sensory therapeutic interventions. Remote psychological treatments, such as hypnosis or guided meditation, typically lack personalized, data-driven adjustments based on the patient's real-time physiological data, thereby making it difficult to achieve precise synergy between the mind and body during treatment. The absence of real-time data integration reduces the potential effectiveness of remote interventions and limits the scope of their therapeutic applications.

Furthermore, most remote systems do not provide comprehensive solutions for long-term health management. Chronic disease management and psychological health improvement require continuous, integrated monitoring of both physical and mental health parameters. Current solutions fail to integrate such monitoring with advanced therapies in a seamless, automated way, thereby leaving patients to rely on separate devices or healthcare providers to manage different aspects of their health. Additionally, conventional approaches to remote mind-body synergy lack integration with modern technologies, such as artificial intelligence and real-time data processing, which are critical to optimizing patient outcomes and ensuring personalized, dynamic care.

To address these limitations, there is a need for a multi-functional hyperbaric oxygen device that integrates hyperbaric oxygen therapy, light therapy, sleep assistance, and temperature regulation with a remote mind-body synergy system for chronic disease recovery, psychological health improvement, and comprehensive health management. There is also a need for a multi-functional hyperbaric oxygen device that allows users to receive personalized, data-driven therapies, adjusting oxygen levels, light wavelengths, and temperature settings based on real-time physiological and psychological monitoring. There is also a need for a multi-functional hyperbaric oxygen device that enables users to conduct therapy sessions from the comfort of their home, thereby minimizing the need for frequent visits to specialized medical centers, and improving accessibility for those with mobility challenges. Further, there is also a need for a multi-functional hyperbaric oxygen device that ensures patient safety by incorporating automated oxygen regulation, CO₂ management, and safety sensors to prevent risks associated with excessive oxygen exposure and ensure optimal therapeutic conditions.

### SUMMARY OF THE INVENTION

The following presents a simplified summary of one or more embodiments of the present disclosure to provide a basic understanding of such embodiments. This summary is not an extensive overview of all contemplated embodiments and is intended to neither identify key nor critical elements of all embodiments nor delineate the scope of any or all embodiments.

The present disclosure, in one or more embodiments, relates to a multi-functional hyperbaric oxygen device that integrates hyperbaric oxygen therapy, light therapy, sleep assistance, and temperature regulation with a remote mind-body synergy system for chronic disease recovery, physiological and psychological health improvement, and comprehensive health management. The multi-functional hyperbaric oxygen device allows users to receive personalized, data-driven therapies, adjusting oxygen levels, light wavelengths, and temperature settings based on real-time physiological and psychological monitoring. The multi-functional hyperbaric oxygen device enables users to conduct therapy sessions from the comfort of their home, thereby minimizing the need for frequent visits to specialized medical centers, and improving accessibility for those with mobility challenges. The multi-functional hyperbaric oxygen device ensures patient safety by incorporating automated oxygen regulation, CO₂ management, and safety sensors to prevent risks associated with excessive oxygen exposure and ensure optimal therapeutic conditions.

In one embodiment herein, the multi-functional hyperbaric oxygen device comprises a housing, a hyperbaric oxygen therapy unit, a light therapy unit, a temperature regulation unit, a remote mind-body synergy system, and a control system. In one embodiment herein, the housing defines a treatment chamber. In one embodiment herein, the hyperbaric oxygen therapy unit is disposed within the treatment chamber. The hyperbaric oxygen therapy unit is configured to deliver controlled oxygen levels to a user in the treatment chamber.

In one embodiment herein, the hyperbaric oxygen therapy unit comprises an oxygen management system that is configured to continuously monitor the oxygen levels within the multi-functional hyperbaric oxygen device, thereby dynamically adjusting the oxygen supply to maintain safe levels. The oxygen management system is further configured to automatically regulate the oxygen levels by switching to low-concentration modes after a predefined duration, thereby preventing prolonged exposure to high oxygen levels and ensuring user safety. The oxygen management system is further configured to automatically activate an air circulation mode to restore and maintain a normal oxygen balance within the multi-functional hyperbaric oxygen device during emergency situations.

In one embodiment herein, the light therapy unit is integrated within the treatment chamber. The light therapy unit is configured to emit light at predetermined wavelengths to promote physiological healing and enhance psychological health. The light therapy unit comprises a light intensity management system that is configured to adjust lighting within the treatment chamber in synchronization with day and night cycles, thereby promoting optimal circadian rhythm alignment and enhancing the user's sleep quality. In one embodiment herein, the temperature regulation unit is disposed within the treatment chamber. The temperature regulation unit is configured to regulate and maintain a predefined temperature range to optimize therapeutic conditions within the treatment chamber.

In one embodiment herein, the remote mind-body synergy system is integrated into the multi-functional hyperbaric oxygen device. The remote mind-body synergy system is configured to monitor real-time physiological and psychological parameters of the user, transmit the monitored data to a healthcare specialist for feedback, and facilitate generation of a personalized treatment plan tailored to the user's needs.

In one embodiment herein, the remote mind-body synergy system comprises a computing device having a processor and a memory to store one or more instructions executable by the processor. The computing device is in communication with a server via a network. The computing device is configured to execute a plurality of modules, which includes a physiological monitoring module, a psychological monitoring module, a data transmission module, and a processing module.

In one embodiment herein, the physiological monitoring module is configured to collect and analyze real-time physiological data of a user in the multi-functional hyperbaric oxygen device, such as heart rate, blood oxygen levels, respiration rate, and body temperature. In one embodiment herein, the psychological monitoring module is configured to assess and analyze psychological data of the user, such as stress levels, emotional state, and cognitive performance.

In one embodiment herein, the data transmission module is configured to transmit the collected physiological and psychological data to the server for analysis by the healthcare specialist, thereby enabling the healthcare specialist to provide feedback. In one embodiment herein, the processing module is configured to receive the feedback and generate a personalized treatment plan for the user using one or more artificial intelligence (AI)-based models to optimize therapeutic outcomes.

In one embodiment herein, the physiological monitoring module and the psychological monitoring module are configured to receive data from one or more sensors, which continuously monitor the real-time physiological and psychological parameters of the user, thereby enabling the remote mind-body synergy system to analyze and generate health reports.

In one embodiment herein, the plurality of modules comprises a communication module that is configured to enable real-time interaction between the user and the healthcare specialist via at least one of audio, video, and text-based communication, thereby facilitating immediate feedback, personalized guidance, and real-time adjustments to the user's treatment plan. The communication module is in communication with a capturing unit and a microphone, which enable two-way audio and video communication between the user and the healthcare specialist for real-time interaction and monitoring.

In one embodiment herein, the plurality of modules comprises a data security module that is configured to employ advanced encryption technologies for safeguarding user privacy and ensuring secure data transmission, and support distributed storage systems for reliable and efficient data management.

In one embodiment herein, the control system is in communication with the remote mind-body synergy system. The control system is configured to adjust therapy settings based on the feedback provided by the healthcare specialist, thereby optimizing therapeutic experience for chronic disease recovery, psychological health improvement, and overall health management of the user. In one embodiment herein, the multi-functional hyperbaric oxygen device comprises one or more carbon dioxide discharge devices that are configured to remove excess carbon dioxide from the multi-functional hyperbaric oxygen device, thereby preventing buildup in a high-pressure environment.

In one embodiment herein, the multi-functional hyperbaric oxygen device comprises an alarm system that is configured to continuously monitor environmental and operational conditions within the multi-functional hyperbaric oxygen device, and triggers an audible and visual alarm upon detecting abnormal conditions, such as deviations in the oxygen levels, temperature, and user health parameters, thereby ensuring safety and timely intervention. In one embodiment herein, the multi-functional hyperbaric oxygen device comprises a sound system that is configured to play therapeutic audio content, such as calming music and guided relaxation sessions, to reduce stress, alleviate anxiety, and enhance user's overall relaxation and therapeutic experience.

In one embodiment herein, the multi-functional hyperbaric oxygen device comprises a UV sterilization unit that is configured to automatically disinfect interior surfaces of the multi-functional hyperbaric oxygen device after each use using a UVC ultraviolet light. In one embodiment herein, the multi-functional hyperbaric oxygen device comprises a high-density memory foam mattress that is configured to contour to the user's body, thereby providing enhanced comfort and support, and suitable for long-term use.

According to an aspect, a method is disclosed for operating the remote mind-body synergy system integrated into the multi-functional hyperbaric oxygen device. First, at one step, the physiological monitoring module collects and analyzes the real-time physiological data of the user in the multi-functional hyperbaric oxygen device, such as heart rate, blood oxygen levels, respiration rate, and body temperature. At another step, the psychological monitoring module assesses and analyzes the psychological data of the user, such as stress levels, emotional state, and cognitive performance.

At another step, the data transmission module transmits the collected physiological and psychological data to the healthcare specialist for feedback. Further, at another step, the processing module receives the feedback and generates the personalized treatment plan for the user using one or more artificial intelligence (AI)-based models to optimize therapeutic outcomes.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1 illustrates a perspective view of a multi-functional hyperbaric oxygen device, in accordance with embodiments of the invention.

FIG. 2 illustrates an isometric view of the multi-functional hyperbaric oxygen device, in accordance with embodiments of the invention.

FIG. 3 illustrates a block diagram of the multi-functional hyperbaric oxygen device, in accordance with embodiments of the invention.

FIG. 4 illustrates a block diagram of a remote mind-body synergy system integrated into the multi-functional hyperbaric oxygen device, in accordance with embodiments of the invention.

FIG. 5 illustrates a front view of another embodiment of the multi-functional hyperbaric oxygen device, in accordance with embodiments of the invention.

FIG. 6 illustrates a rear view of another embodiment of the multi-functional hyperbaric oxygen device, in accordance with embodiments of the invention.

FIG. 7 illustrates a flowchart of a method for operating the remote mind-body synergy system integrated into the multi-functional hyperbaric oxygen device, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

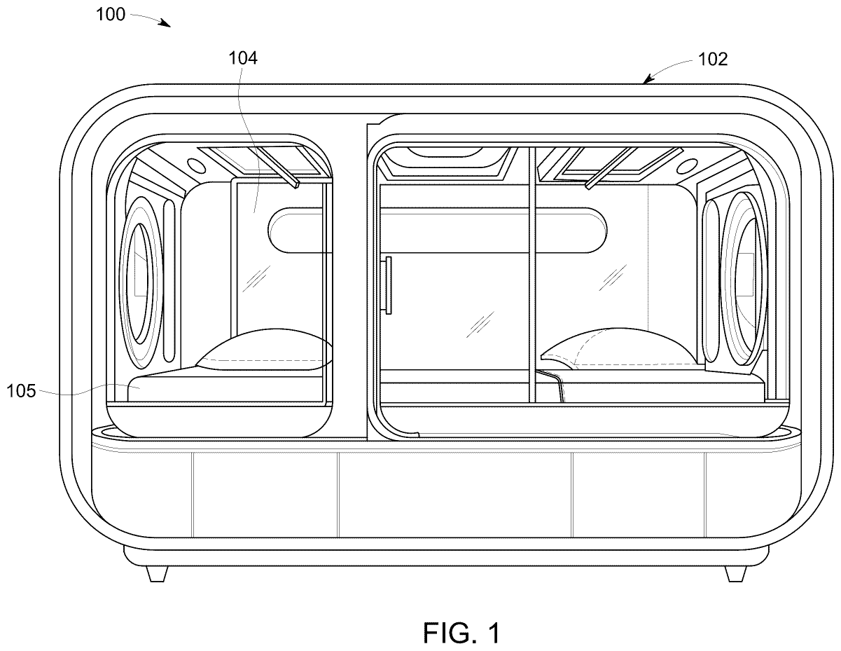

FIG. 1 refers to a perspective view of a multi-functional hyperbaric oxygen device 100. In one embodiment herein, the multi-functional hyperbaric oxygen device 100 integrates hyperbaric oxygen therapy, light therapy, sleep assistance, and temperature regulation with a remote mind-body synergy system for chronic disease recovery, physiological and psychological health improvement, and comprehensive health management. The multi-functional hyperbaric oxygen device 100 allows users to receive personalized, data-driven therapies, adjusting oxygen levels, light wavelengths, and temperature settings based on real-time physiological and psychological monitoring. The multi-functional hyperbaric oxygen device 100 enables users to conduct therapy sessions from the comfort of their home, thereby minimizing the need for frequent visits to specialized medical centers, and improving accessibility for those with mobility challenges. The multi-functional hyperbaric oxygen device 100 ensures patient safety by incorporating automated oxygen regulation, CO₂ management, and safety sensors to prevent risks associated with excessive oxygen exposure and ensure optimal therapeutic conditions.

In one embodiment herein, the multi-functional hyperbaric oxygen device 100 comprises a housing 102, which defines a treatment chamber 104 that encloses all essential components, thereby providing a controlled and safe therapeutic environment. The compact and curvilinear design of the housing 102 ensures ergonomic usability and maximizes comfort for the user during extended therapy sessions. The treatment chamber 104 is configured with temperature regulation and a soundproof design to create a quiet and comfortable sleeping environment, thereby promoting deep and restful sleep. The pressure within the treatment chamber 104 is maintained at 1.3 ATA, utilizing a dual-layer sealing structure to ensure airtightness and enhance operational safety.

In one embodiment herein, the treatment chamber 104 is equipped with a high-density memory foam mattress 105 to provide optimal comfort. This foam mattress 105 contours to the user's body, thereby offering exceptional support and comfort during therapy. The foam mattress's 105 durability and design make it ideal for long-term use, thereby enhancing the user's experience. The foam mattress 105 comprises breathable and washable mattress covers for easy cleaning and maintenance.

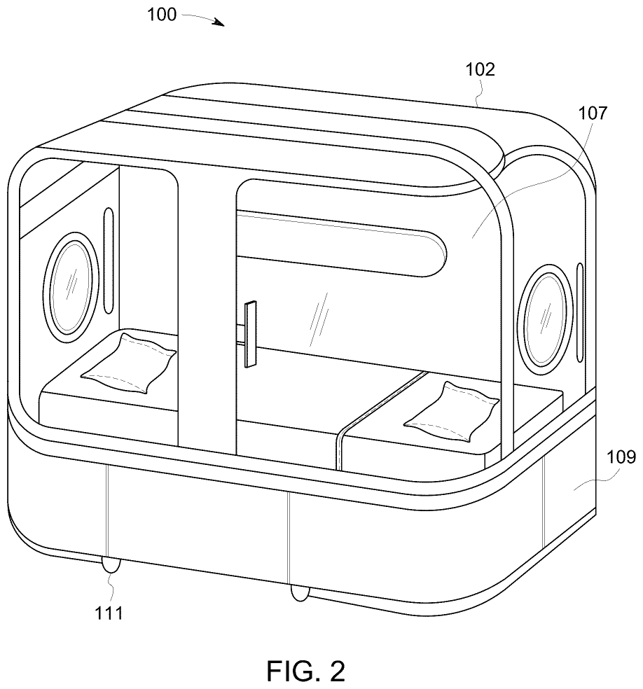

FIG. 2 refers to an isometric view of the multi-functional hyperbaric oxygen device 100. In one embodiment herein, the multifunctional hyperbaric oxygen device 100 comprises a rectangular structure with a rounded top and reinforced frame. The multi-functional hyperbaric oxygen device 100 is designed with multiple transparent or semi-transparent panels 107, which may be made from durable materials to allow visibility while ensuring safety during operation. In one embodiment herein, the multi-functional hyperbaric oxygen device 100 comprises a sturdy base 109 equipped with small wheels 111 to enable mobility for convenient repositioning.

In one embodiment herein, the multi-functional hyperbaric oxygen device 100 features a spacious design, which ensures user comfort during operation. For example, the treatment chamber 104 for a single-user can be designed with measurements 2000 mm by 1000 mm by 1200 mm, thereby offering ample room for reclining or sitting upright. Similarly, the treatment chamber 104 for a double-user can be designed with measurements 2000 mm by 1500 mm by 1400 mm, thereby providing sufficient space to accommodate two users comfortably, with options for either reclining or sitting upright, depending on the therapeutic requirements.

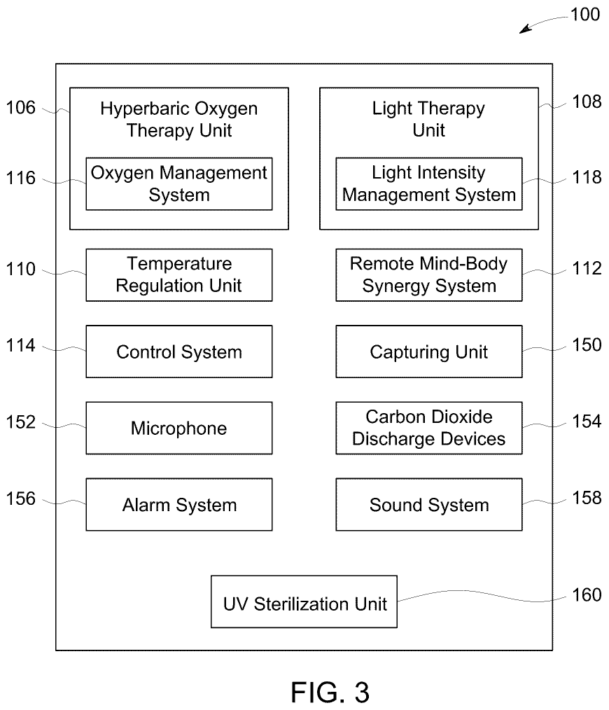

FIG. 3 refers to a block diagram of the multi-functional hyperbaric oxygen device 100. In one embodiment herein, the multi-functional hyperbaric oxygen device 100 further comprises a hyperbaric oxygen therapy unit 106, a light therapy unit 108, a temperature regulation unit 110, a remote mind-body synergy system 112, and a control system 114. In one embodiment herein, the hyperbaric oxygen therapy unit 106 is positioned within the treatment chamber 104. The hyperbaric oxygen therapy unit 106 is configured to deliver controlled oxygen levels tailored to the user's specific therapeutic needs. The hyperbaric oxygen therapy unit 106 enhances the body's oxygen absorption by providing high-pressurized oxygen, thereby promoting faster recovery from injuries, chronic diseases, and improving overall cellular health. The hyperbaric oxygen therapy unit 106 is operable within a pressure range of 1.1 to 1.4 atmospheres.

In one embodiment herein, the hyperbaric oxygen therapy unit 106 further includes an oxygen management system 116 that continuously monitors oxygen levels within the treatment chamber 104. This oxygen management system 116 dynamically adjusts the oxygen supply to maintain safe levels and prevent the risk of oxygen toxicity. Additionally, the oxygen management system 116 features an automatic low-concentration mode that activates after a predefined time period to avoid prolonged exposure to high oxygen levels. During emergencies, the oxygen management system 116 can initiate an air circulation mode to restore normal oxygen balance, thereby ensuring user safety and operational stability.

In one embodiment herein, the light therapy unit 108 is integrated within the treatment chamber 104. The light therapy unit 108 emits light at particular wavelengths to promote physiological healing and enhance psychological well-being. The light therapy unit 108 also provides infrared therapy that enhances blood circulation and muscle relaxation, thereby relieving fatigue caused by sedentary lifestyles. The light therapy unit 108 includes a light intensity management system 118 that adjusts brightness and color temperature of the lighting to mimic natural day-night cycles. This synchronization with the user's circadian rhythms improves sleep quality, supports mental clarity, and fosters overall health improvements.

In one embodiment herein, the temperature regulation unit 110 is situated within the treatment chamber 104. The temperature regulation unit 110 is configured to maintain a predefined temperature range conducive to therapy. By providing a stable and controlled thermal environment, the temperature regulation unit 110 optimizes the overall effectiveness of hyperbaric oxygen therapy, thereby ensuring user comfort and relaxation throughout the session. In one embodiment, the temperature regulation unit 110 includes advanced temperature control and ventilation systems designed to maintain a comfortable environment within the treatment chamber 104 for the user, regardless of seasonal conditions, whether during summer heat or winter cold.

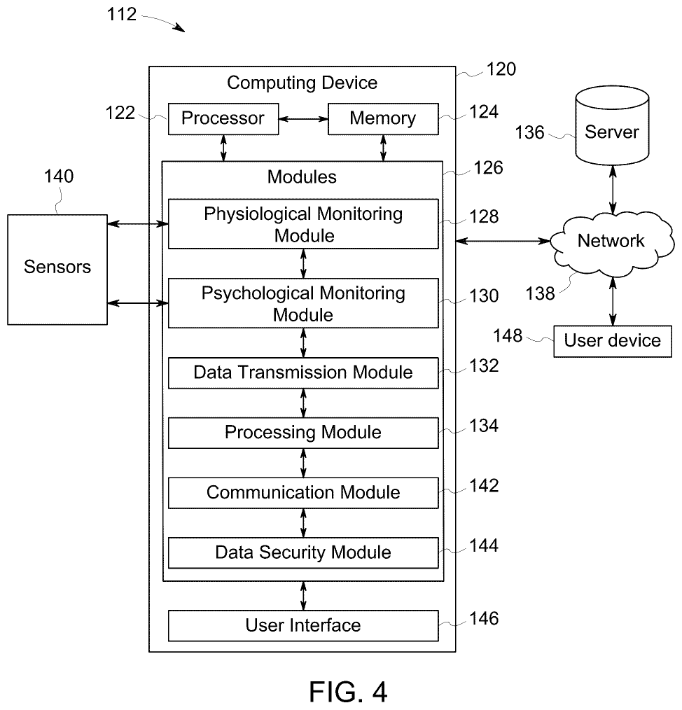

FIG. 4 refers to a block diagram of the remote mind-body synergy system 112. In one embodiment herein, the remote mind-body synergy system 112 is integrated seamlessly into the multi-functional hyperbaric oxygen device 100. This remote mind-body synergy system 112 is configured to monitor the user's real-time physiological and psychological parameters and transmit data to a healthcare specialist for analysis. By leveraging this data, the remote mind-body synergy system 112 facilitates the development of a personalized treatment plan tailored to the user's health profile and therapeutic needs.

In one embodiment herein, the remote mind-body synergy system 112 comprises a computing device 120 equipped with a processor 122 and a memory 124. This computing device 120 executes a plurality of modules 126, such as a physiological monitoring module 128, a psychological monitoring module 130, a data transmission module 132, and a processing module 134. The computing device 120 communicates with a server 136 via a network 138, thereby enabling efficient data exchange and analysis.

In one embodiment herein, the physiological monitoring module 128 collects real-time physiological data such as heart rate, blood oxygen levels, respiration rate, and body temperature. This data provides a comprehensive overview of the user's physical health during therapy. In one embodiment herein, the psychological monitoring module 130 assesses the user's mental and emotional state by analyzing data related to stress levels, cognitive performance, and emotional well-being. This dual focus ensures a holistic approach to therapy, thereby addressing both physical and mental health.

In one embodiment herein, the data transmission module 132 transmits the collected data to the server 136, where it can be analyzed by the healthcare specialist using a user device 148. This enables a professional evaluation of the user's condition and provides a feedback. In one embodiment herein, the processing module 134 uses artificial intelligence (AI)-based models to analyze the feedback provided by the healthcare specialist, and generate the personalized treatment plan. By leveraging advanced machine learning models, the remote mind-body synergy system 112 ensures precision and customization in its recommendations, thereby optimizing therapeutic outcomes. In one embodiment herein, the user device 148 can be, but not limited to, a smartphone, a laptop, a computer, a tablet, and thereof.

In one embodiment herein, the remote mind-body synergy system 112 comprises one or more sensors 140, which continuously monitor the user's real-time parameters, thereby providing consistent and accurate data for analysis. This functionality allows the remote mind-body synergy system 112 to generate detailed health reports, which enable informed decision-making by the healthcare specialist.

In one embodiment herein, the modules 126 further includes a communication module 142 that supports real-time interaction between the user and the healthcare specialist. The communication module 142 facilitates immediate feedback, guidance, and adjustments to the user's treatment plan using audio, video, and text-based communication. In one embodiment herein, the multi-functional hyperbaric oxygen device 100 further comprises a capturing unit 150 and a microphone 152 that are configured to enable two-way communication, thereby ensuring seamless interaction and monitoring. In one embodiment herein, the capturing unit 150 can be a high-definition camera. In one embodiment herein, the modules further comprises a data security module 144 that employs advanced encryption technologies, such as AES-256 encryption, to safeguard user privacy and data security. This data security module 144 ensures secure data transmission and supports distributed storage systems, thereby providing reliability and efficiency in data management.

Referring to FIG. 3, the control system 114 works in tandem with the remote mind-body synergy system 112 to adjust therapy settings based on the feedback provided by the healthcare specialist. This dynamic control enhances therapeutic outcomes, which make the multi-functional hyperbaric oxygen device 100 suitable for chronic disease recovery, psychological health improvement, and overall wellness management. In one embodiment herein, the multi-functional hyperbaric oxygen device 100 further includes one or more carbon dioxide discharge devices 154 that are configured to remove excess carbon dioxide from the treatment chamber 104. This feature prevents the buildup of carbon dioxide in the high-pressure environment, thereby maintaining a safe and comfortable atmosphere for the user.

In one embodiment herein, the multi-functional hyperbaric oxygen device 100 further comprises an alarm system 156 that is configured to continuously monitor the environmental and operational conditions of the multi-functional hyperbaric oxygen device 100. In case of abnormal situations such as oxygen level deviations, temperature fluctuations, or irregular user health parameters, the alarm system 156 triggers audible and visual alarms to ensure timely intervention and user safety. In one embodiment herein, the multi-functional hyperbaric oxygen device 100 further comprises a sound system 158 that is configured to play therapeutic audio content, such as calming music, and guided relaxation and hypnosis sessions. These features reduce stress and anxiety in the user, thereby enhancing the overall relaxation and therapeutic experience for the user.

In one embodiment herein, the multi-functional hyperbaric oxygen device 100 further comprises a UV sterilization unit 160 that is configured to employ an UVC ultraviolet light to disinfect interior surfaces of the treatment chamber 104 after each use. This automatic sterilization process ensures a hygienic environment for every therapy session, thereby maintaining high safety standards.

In one embodiment herein, the operation of the multi-functional hyperbaric oxygen device 100 begins with the user entering the treatment chamber 104. The user is guided to ensure that they are seated or positioned comfortably within the treatment chamber 104. The control system 114, which is integrated within the multi-functional hyperbaric oxygen device 100, is initially activated by the user via a user interface 146 or remote control. Once the control system 114 is activated to be operational, the one or more sensors 140 are activated and continuously track the physiological parameters, such as heart rate, blood oxygen levels, respiration rate, and body temperature, and the psychological parameters such as stress levels and mental state. This monitored data is collected by the physiological monitoring module 128 and the psychological monitoring module 130 of the remote mind-body synergy system 112. Then, the data transmission module 144 transmits the collected data to the server 136 for analysis by the healthcare specialist, thereby enabling the healthcare specialist to provide feedback using the user device 148.

In one embodiment herein, the processing module 134 receives the feedback and generates the personalized treatment plan for the user using one or more artificial intelligence (AI)-based models to optimize therapeutic outcomes. Simultaneously, the feedback provided by the healthcare specialist is transmitted to the control system 114, which adjusts the therapy settings, such as oxygen concentration levels, light intensity, and temperature, in response to the user's ongoing physiological and psychological needs.

In one embodiment herein, the hyperbaric oxygen therapy unit 106 provides controlled oxygen levels to the user. Initially, the multi-functional hyperbaric oxygen device 100 adjusts the oxygen supply to the optimal concentration based on the user's personalized treatment plan. The oxygen management system 116 continuously monitors the oxygen levels within the treatment chamber 104, thereby ensuring that they remain within a safe range. If a predefined time threshold is reached, the oxygen management system 116 automatically adjusts the oxygen supply to a lower concentration to prevent prolonged exposure to high oxygen levels. This ensures the user's safety throughout the therapy session.

Concurrently, the light therapy unit 108 emits therapeutic light at specific wavelengths, calibrated to promote healing and mental well-being. The light intensity management system 118 ensures that the light output is adjusted to synchronize with the user's circadian rhythm, thereby enhancing the overall therapeutic effects. The light therapy settings, such as brightness and color temperature, are automatically optimized based on the time of day and the user's health profile.

In one embodiment herein, the temperature regulation unit 110 is responsible for maintaining an optimal temperature within the treatment chamber 104. The temperature is set according to the user's preferences and the treatment requirements, thereby providing a comfortable and therapeutic environment. The temperature regulation unit 110 works in tandem with the hyperbaric oxygen therapy unit 106 and the light therapy unit 108 to ensure a holistic and integrated therapeutic experience.

During the session, the user is constantly monitored by the one or more sensors 140. The physiological and psychological data is collected by the physiological monitoring module 128 and the psychological monitoring module 130, and then transmitted to the healthcare specialist via the data transmission module 132. This allows the healthcare specialist to observe the user's progress in real-time and make adjustments to the personalized treatment plan if necessary. If any unusual readings are detected, such as an abnormal heart rate or oxygen levels, the healthcare specialist can intervene by adjusting the therapy settings remotely, thereby providing immediate feedback and personalized guidance.

In one embodiment herein, the communication module 142 is activated to enable two-way communication between the user and the healthcare specialist. The user can interact with the healthcare specialist via audio, video, or text-based communication, depending on their preferences and the severity of the situation. The capturing unit 150 and the microphone 152 are utilized for clear audio and video exchange. This real-time interaction enhances the personalized nature of the treatment, thereby allowing the user to ask questions, receive feedback, and ensure the therapy is proceeding as intended.

As the therapy session progresses, the control system 114 continuously evaluates the user's data, thereby making dynamic adjustments to oxygen concentration, light therapy settings, and temperature. These adjustments are aimed at optimizing the user's therapeutic experience and improving outcomes such as chronic disease recovery, mental health improvements, and general well-being. The control system 114 ensures that the therapy remains adaptive, thereby continuously fine-tuning the user's personalized treatment plan based on real-time data.

At any point during the session, if the control system 114 detects an emergency or a deviation from normal parameters, such as a drop in oxygen levels or an increase in carbon dioxide concentration, the oxygen management system 116 immediately activates the air circulation mode. This air circulation mode restores the oxygen balance by releasing excess carbon dioxide and ensuring that the oxygen levels remain within safe limits. If required, the alarm system 156 triggers audible and visual alerts to notify both the user and the healthcare specialist of the issue, thereby ensuring timely intervention.

After the session is completed, the user is prompted to exit the treatment chamber 104. The control system 114 automatically activates the UV sterilization unit 160 to disinfect the interior surfaces of the treatment chamber 104. This ensures that any residual contaminants or bacteria are eliminated before the next use. The high-density memory foam mattress 105 is also designed to maintain its shape and comfort for the next user, thereby contributing to the overall hygiene of the multi-functional hyperbaric oxygen device 100.

Once the user exits the treatment chamber 104, a final set of data may be collected, such as feedback on the user's subjective experience of the session, any discomfort or positive effects observed, and overall satisfaction. This feedback can be transmitted to the healthcare specialist and stored for future reference, which contributes to the ongoing refinement of the user's treatment plan.

In the event of any technical issues or system errors during operation, the control system 114 may perform a self-diagnosis, identifying and alerting the user to any faults. The control system 114 may also provide troubleshooting instructions or prompt the user to contact technical support for resolution. This ensures that the multi-functional hyperbaric oxygen device 100 remains reliable and user-friendly, providing a seamless and effective therapeutic experience for all users.

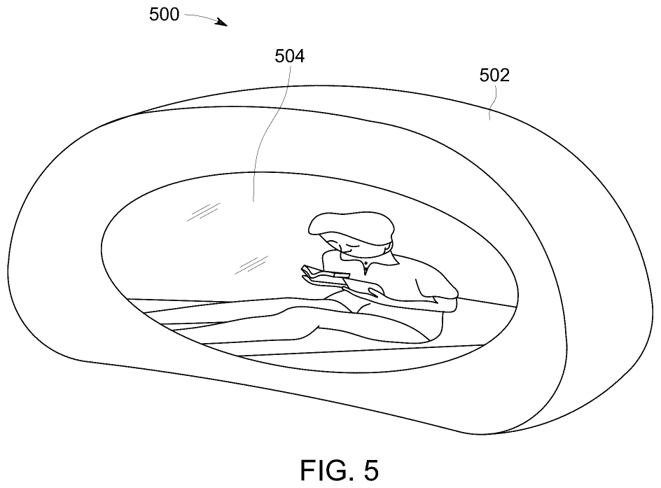

FIG. 5 refers to a front view of another embodiment of the multi-functional hyperbaric oxygen device 500. In one embodiment herein, the multi-functional hyperbaric oxygen device 500 comprises a streamlined, cylindrical-shaped enclosure 502 designed to accommodate a single person. The outer structure has a smooth, curved surface, giving it a futuristic and ergonomic appearance. The multi-functional hyperbaric oxygen device 500 comprises a large, oval-shaped window 504 on one side, which provides visibility into the interior, thereby allowing natural light to enter while offering an enclosed environment. The interior can be spacious enough for reclining and provides a sense of privacy, which enhances comfort and usability. The minimalistic design implies potential applications in therapeutic or wellness devices, thereby emphasizing user-centric functionality and aesthetic appeal.

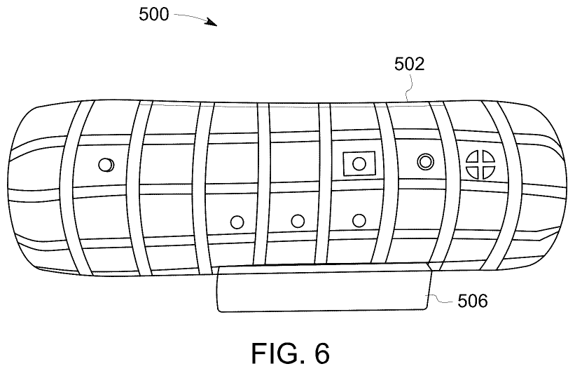

FIG. 6 refers to a rear view of another embodiment of the multi-functional hyperbaric oxygen device 500. In one embodiment herein, the surface of the multifunctional hyperbaric oxygen device 500 includes circular and square openings or panels, which may serve various functional purposes, such as ventilation, control access, or monitoring. The multi-functional hyperbaric oxygen device 500 is mounted on a rectangular base 506, ensuring stability and ease of installation. This embodiment emphasizes a robust and modular design, suitable for diverse applications while maintaining the core functionality of delivering controlled hyperbaric oxygen therapy.

Overall, the multi-functional hyperbaric oxygen device 100 integrates multiple health-optimization technologies, which significantly enhance its utility and appeal compared to traditional hyperbaric chambers. One of the key advantages is the ability of the multi-functional hyperbaric oxygen device 100 to function as a bed replacement. The spacious design, comfortable memory foam mattress, and integrated aesthetic construction of the treatment chamber 104 provide the users with a more comfortable and versatile solution for sleep and recovery. By incorporating temperature regulation, soundproofing, and intelligent light adjustment, the multi-functional hyperbaric oxygen device 100 optimizes circadian rhythms, thereby promoting enhanced rest and overall well-being.

Another advantage of the multi-functional hyperbaric oxygen device 100 is its dynamic oxygen concentration regulation. The oxygen management system 116 continuously monitors and adjusts oxygen levels to prevent oxygen toxicity. Automated modes are incorporated to adjust the oxygen concentration during sleep and at specific intervals, thereby ensuring safe, long-term use. Additionally, the integrated carbon dioxide discharge systems 154 and the alarm system 156 enhance user safety by mitigating potential risks associated with oxygen therapy in the multi-functional hyperbaric oxygen device 100.

The multi-functional hyperbaric oxygen device 100 is distinguished by its integration of various therapeutic modalities, such as hyperbaric oxygen therapy, light therapy, infrared therapy, and sleep-assistance functions. This multi-therapy approach provides comprehensive health management, thereby promoting physical recovery, relaxation, and mental well-being. For instance, the high-pressure oxygen environment accelerates cellular metabolism and repair, while the infrared therapy promotes blood circulation and muscle relaxation. Additionally, the ability of the multi-functional hyperbaric oxygen device 100 to play calming music and provide a soothing lighting environment to reduce stress and anxiety, thereby making it suitable for holistic wellness applications.

Furthermore, the smart monitoring and data encryption capabilities of the multi-functional hyperbaric oxygen device 100 facilitate real-time health data collection while ensuring the privacy and security of users' physiological information. This data-driven approach enables personalized health management and remote monitoring, thereby making the multi-functional hyperbaric oxygen device 100 suitable for both home use and commercial environments, such as wellness centers or corporate health programs. The integrated UV sterilization unit 160 provides effective interior disinfection and chemical-free cleaning, thereby ensuring the multi-functional hyperbaric oxygen device 100 remains user-friendly and environmentally conscious. The combination of advanced safety features, functionality, and holistic health support makes the multi-functional hyperbaric oxygen device 100 a groundbreaking and practical solution for enhancing health, sleep, and overall quality of life.

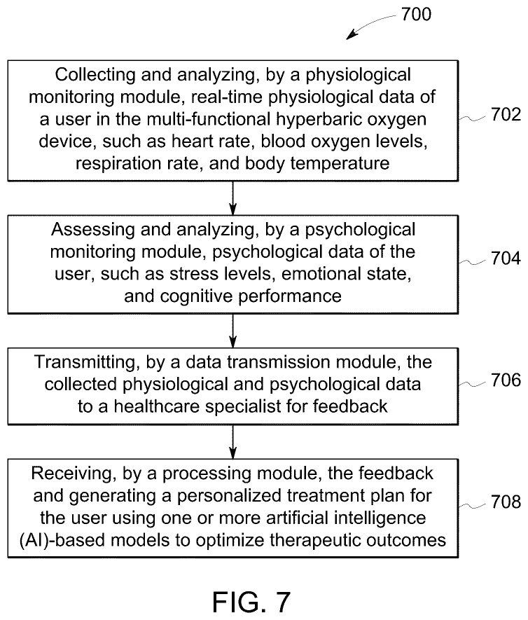

FIG. 7 refers to a flowchart 700 of a method for operating the remote mind-body synergy system 112 integrated into the multi-functional hyperbaric oxygen device 100. First, at step 702, the physiological monitoring module 128 collects and analyzes the real-time physiological data of the user in the multi-functional hyperbaric oxygen device 100, such as heart rate, blood oxygen levels, respiration rate, and body temperature. At step 704, the psychological monitoring module 130 assesses and analyzes the psychological data of the user, such as stress levels, emotional state, and cognitive performance. At step 706, the data transmission module 132 transmits the collected physiological and psychological data to the healthcare specialist for feedback. Further, at step 708, the processing module 134 receives the feedback and generates the personalized treatment plan for the user using one or more artificial intelligence (AI)-based models to optimize therapeutic outcomes.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. A multi-functional hyperbaric oxygen device, comprising:
a housing defining a treatment chamber;
a hyperbaric oxygen therapy unit disposed within the treatment chamber, wherein said hyperbaric oxygen therapy unit is configured to deliver controlled oxygen levels to a user within the treatment chamber;
a light therapy unit integrated within the treatment chamber, wherein said light therapy unit is configured to emit lights at predetermined wavelengths to promote physiological healing and enhance psychological health of the user;
a temperature regulation unit disposed within the treatment chamber, wherein said temperature regulation unit is configured to regulate and maintain a predefined temperature range to optimize therapeutic conditions within the treatment chamber;
a remote mind-body synergy system integrated into the multi-functional hyperbaric oxygen device, wherein said remote mind-body synergy system is configured to monitor real-time physiological and psychological parameters of the user, transmit monitored data to a healthcare specialist for obtaining feedback, and facilitate generation of a personalized treatment plan tailored to the user's needs; and
a control system adapted to communicate with the remote mind-body synergy system, wherein said control system is configured to adjust therapy settings based on the feedback, thereby optimizing therapeutic experience for chronic disease recovery, psychological health improvement, and overall health management of the user.

2. The multi-functional hyperbaric oxygen device of claim 1, wherein said hyperbaric oxygen therapy unit comprises an oxygen management system that is configured to continuously monitor the oxygen levels within the treatment chamber, thereby dynamically adjusting an oxygen supply to maintain safe levels.

3. The multi-functional hyperbaric oxygen device of claim 2, wherein said oxygen management system is further configured to automatically regulate the oxygen levels by switching to low-concentration modes after a predefined duration, thereby preventing prolonged exposure to high oxygen levels and ensuring user safety.

4. The multi-functional hyperbaric oxygen device of claim 2, wherein said oxygen management system is further configured to automatically activate an air circulation mode to restore and maintain a normal oxygen balance within the treatment chamber during emergency situations.

5. The multi-functional hyperbaric oxygen device of claim 1, wherein said light therapy unit comprises a light intensity management system that is configured to adjust lighting within the treatment chamber in synchronization with day and night cycles, thereby promoting optimal circadian rhythm alignment and enhancing the user's sleep quality.

6. The multi-functional hyperbaric oxygen device of claim 1, wherein said treatment chamber further comprises a capturing unit and a microphone that are configured to enable two-way audio and video communication between the user and the healthcare specialist for real-time interaction and monitoring.

7. The multi-functional hyperbaric oxygen device of claim 1, wherein said treatment chamber comprises one or more carbon dioxide discharge devices that are configured to remove excess carbon dioxide from the multi-functional hyperbaric oxygen device, thereby preventing buildup in a high-pressure environment.

8. The multi-functional hyperbaric oxygen device of claim 1, wherein said treatment chamber comprises an alarm system that is configured to continuously monitor environmental and operational conditions within the multi-functional hyperbaric oxygen device, and trigger an audible and visual alarms upon detecting abnormal conditions, such as deviations in the oxygen levels, temperature, and user health parameters, thereby ensuring safety and timely intervention.

9. The multi-functional hyperbaric oxygen device of claim 1, wherein said treatment chamber comprises a sound system that is configured to play therapeutic audio content, such as calming music and guided relaxation sessions, to reduce stress, alleviate anxiety, and enhance user's overall relaxation and therapeutic experience.

10. The multi-functional hyperbaric oxygen device of claim 1, wherein said remote mind-body synergy system comprises one or more sensors that are configured to continuously monitor the real-time physiological and psychological parameters of the user, thereby enabling the remote mind-body synergy system to analyze and generate health reports.

11. The multi-functional hyperbaric oxygen device of claim 1, wherein said treatment chamber comprises an ultraviolet (UV) sterilization unit that is configured to automatically disinfect interior surfaces of the multi-functional hyperbaric oxygen device after each use through an ultraviolet-C (UVC) light.

12. The multi-functional hyperbaric oxygen device of claim 1, wherein said treatment chamber comprises a high-density memory foam mattress that is configured to contour to the user's body, thereby providing enhanced comfort and support, and suitable for long-term use.

13. A remote mind-body synergy system integrated into a multi-functional hyperbaric oxygen device, comprising:
a computing device having a processor and a memory to store one or more instructions executable by said processor,
wherein said computing device is in communication with a server via a network,
wherein said computing device is configured to execute a plurality of modules, which includes:
a physiological monitoring module configured to collect and analyze real-time physiological data of a user in the multi-functional hyperbaric oxygen device, such as heart rate, blood oxygen levels, respiration rate, and body temperature;
a psychological monitoring module configured to assess and analyze psychological data of the user, such as stress levels, emotional state, and cognitive performance;
a data transmission module configured to transmit the collected physiological and psychological data to the server for analysis by a healthcare specialist, thereby enabling the healthcare specialist to provide feedback; and
a processing module configured to receive the feedback and generate a personalized treatment plan for the user using one or more artificial intelligence (AI)-based models to optimize therapeutic outcomes.

14. The remote mind-body synergy system of claim 13, wherein said physiological monitoring module and the psychological monitoring module are configured to receive data from one or more sensors, which continuously monitor the real-time physiological and psychological parameters of the user, thereby enabling the remote mind-body synergy system to analyze and generate health reports.

15. The remote mind-body synergy system of claim 13, wherein said remote mind-body synergy system is in communication with a control system, which is configured to adjust therapy settings within the multi-functional hyperbaric oxygen device based on the feedback provided by the healthcare specialist, thereby optimizing therapeutic experience for chronic disease recovery, psychological health improvement, and overall health management of the user.

16. The remote mind-body synergy system of claim 13, wherein said plurality of modules comprises a communication module that is configured to enable real-time interaction between the user and the healthcare specialist via at least one of audio, video, and text-based communication, thereby facilitating immediate feedback, personalized guidance, and real-time adjustments to the user's treatment plan.

17. The remote mind-body synergy system of claim 16, wherein said communication module is in communication with a capturing unit and a microphone, which enable two-way audio and video communication between the user and the healthcare specialist for real-time interaction and monitoring.

18. The remote mind-body synergy system of claim 13, wherein said plurality of modules comprises a data security module that is configured to employ advanced encryption technologies for safeguarding user privacy and ensuring secure data transmission, and support distributed storage systems for reliable and efficient data management.

19. A method of operating a remote mind-body synergy system integrated into a multi-functional hyperbaric oxygen device, comprising:
collecting and analyzing, by a physiological monitoring module, real-time physiological data of a user in the multi-functional hyperbaric oxygen device, such as heart rate, blood oxygen levels, respiration rate, and body temperature;
assessing and analyzing, by a psychological monitoring module, psychological data of the user, such as stress levels, emotional state, and cognitive performance;
transmitting, by a data transmission module, the collected physiological and psychological data to a healthcare specialist for feedback; and
receiving, by a processing module, the feedback and generating a personalized treatment plan for the user using one or more artificial intelligence (AI)-based models to optimize therapeutic outcomes.

20. The method of claim 19, wherein the physiological monitoring module and the psychological monitoring module are configured to receive data from one or more sensors, which continuously monitor the real-time physiological and psychological parameters of the user, thereby enabling the remote mind-body synergy system to analyze and generate health reports.
